(19) United States
(12) Patent Application Publication
(10) Pub. No.: US 2005/0233007 A1
(43) Pub. Date: Oct. 20, 2005

(54) Materials and methods of using calcium for reduction of inflammation

(76) Inventors: Eric T. Gugger, Rogers, MN (US); Carolyn Good, Minnetonka, MN (US); Ellen M. Silva, Minnetonka, MN (US)

Correspondence Address: GENERAL MILLS, INC., P.O. BOX 1113, MINNEAPOLIS, MN, 55440, US

(21) Appl. No.: 11/105,953

(22) Filed: Apr. 14, 2005

Related U.S. Application Data
(60) Provisional application No. 60/563,337, filed on Apr. 19, 2004.

(51) Int. Cl. A61K 33/06

(52) U.S. Cl. 424/682

(57) ABSTRACT

Nutritional approaches for the control of inflammation can involve the administration of therapeutic amounts of calcium. The administration of appropriate amounts of calcium to an individual results in a reduction in inflammatory processes and markers associated with the inflammatory processes. The therapeutic amount of calcium can be selected to change a level of inflammation in the individual. The approach for reducing inflammation can comprise a step of measuring the C-reactive protein marker level and administering calcium in amounts selected to induce a change in the C-reactive protein level. The calcium can be incorporated into a product with corresponding instructions to induce a desired inflammation reduction.

This application claims the benefit of priority under 35 U.S.C. 119 (e)(1) of a provisional patent application Ser. No. 60/563,337, filed Apr. 19, 2004, which is incorporated herein by reference in its entirety.

### FIELD OF THE INVENTION

The invention is related to the field of nutrition and nutritional products pertaining to uses of calcium for the reduction of inflammation and inflammation markers, including the C-reactive protein marker.

### BACKGROUND

Calcium plays an active role in many physiological processes. Calcium is the most abundant mineral in the body, and is implicated in many biological controls in humans. About 99% of total body calcium is concentrated in the skeleton and teeth. A typical adult human body contains about 1000 to 1500 grams of calcium, of which about 6-8 grams are in tissues and about 1 gram is in plasma and extracellular fluid. About 45-50% of the calcium in blood is dissociated as free ions, 40-45% is bound to plasma proteins, and about 10% is complexed with ions such as citrate.

The effects of calcium intake on physiological processes can be affected by the body's regulatory mechanisms and by the way that calcium is absorbed. In fact, the body has complex mechanisms to maintain a homeostasis of calcium in the blood. Calcium absorption can vary enormously between individuals. In the intestine, the absorption of calcium involves both transcellular and paracellular transport. Thus, the administration of calcium and its effects on physiological processes are the subject of active scientific investigation in the field of nutrition.

### SUMMARY

The control and reduction of inflammation is important in numerous physiological conditions. As described herein, the nutritional arts are useful for addressing such conditions. And calcium, and calcium sources such as dairy products, can have a role to play in reducing acute and/or chronic inflammation. Materials and methods are described herein for administration of calcium to a subject to cause a reduction in inflammatory processes, and markers associated with such processes. A variety of inflammatory processes can occur in a subject, including acute, chronic, pathological, and essentially normal processes. The presence of an effective level of calcium in the subject, however, can affect inflammatory processes and reduce the amount of inflammation. Markers associated with an inflammatory process can similarly be affected.

In a first aspect, the invention pertains to a method of modifying a biological condition of a subject that comprises administering a therapeutic amount of calcium to the subject. The therapeutic amount of calcium can be selected to change a level of inflammation in the subject.

In another aspect, the invention pertains to a method to reduce inflammation of a subject based on C-reactive protein (“CRP”) levels in the subject. Specifically, the method comprises measuring the CRP level in the subject and administering calcium to the subject. The administration of calcium induces a change in the CRP level.

In a further aspect, the invention pertains to a kit for the modification of a biological condition of a subject. The kit comprises a source of calcium and instruction for administering the source of calcium to change a level of inflammation in the subject.

### DETAILED DESCRIPTION OF EMBODIMENTS

Certain embodiments are described, below, in the context of other information in the art to provide a suitable context for the presentation of therapeutic methodologies and materials. In the past, a variety of treatments have been developed to control inflammation, including methods using proteins, small molecule drugs, and antibodies. Such treatments have focused on treating specific inflammatory conditions or targeted specific types of receptors. It has not been previously appreciated, however, that a treatment to change inflammation levels can help to prevent many pathologies caused by inflammation processes. Materials and methods for administration of calcium to a subject have been found that change inflammation levels, and the levels of inflammation markers. Moreover, materials and methods for administration of calcium have been found that tend to reduce the amount of adipose tissue, including trunkal or visceral adipose tissue, which have been implicated in the production of inflammatory agents.

Methods according to embodiments of the invention are provided for controlling and reducing inflammation levels by administering calcium. Without being bound to a particular theory or mechanism of action, effective calcium intake by a subject contributes to activation of metabolic pathways in the subject that produce a reduction in inflammation. Evidence for such pathways includes observations that calcium helps to shift the overall balance of a subject's adiposity away from trunkal and/or visceral adiposity, which may be involved in inflammation. Past studies have correlated calcium intake with a loss of overall body weight, but a mere loss of body weight is not believed to correlate with a reduction of inflammation.

Materials and corresponding methods are also described for using calcium to measure and control inflammation marker levels. Appropriate ways are set forth for controlling calcium intake through dietary approaches and/or supplements that can achieve a desirable amount of inflammation control and possibly weight loss. Acute and/or prophylactic control of inflammation is expected to bring numerous health benefits because inflammation is associated with a wide variety of health conditions. For example, atherosclerosis, a gradual thickening of the arterial wall that is a fundamental process responsible for a majority of cardiovascular diseases, is an inflammatory process (Ross, R., Atherosclerosis—an inflammatory disease. N Engl J Med, 1999.340(2): p. 115-26). Cardiovascular disease is currently asserted as the leading case of death in the United States for both men and women. Inflammation is implicated in a wide variety of other pathological conditions, including rheumatoid arthritis, psoriasis, asthma, systemic lupus erythematosus, and osteoarthritis. Inflammation markers can detect inflammation at some level in a subject at any given time. Therefore the level of inflammation may be a predictor for certain health consequences. Further, the source of inflammation may also be a factor that affects health.

One embodiment of the invention is a method of modifying a biological condition of a subject by administering a therapeutic amount of calcium to the subject, wherein the therapeutic amount of calcium is selected to change a level of inflammation in the subject. The level of inflammation in the subject can be changed in, e.g., a tissue or bodily fluid of the subject. A subject may be, e.g., a human, mammal, animal, patient, or other being that can ingest a calcium source and benefit from a reduction in inflammation.

Another embodiment of the invention is a kit for modifying a biological condition of a subject. The kit may include, e.g., a source of calcium and an instruction for administering the source of calcium to the subject to change a level of inflammation in the subject.

The instruction may include, e.g., a description of administering the source of calcium to the subject to change or decrease the level of inflammation or inflammation marker in the subject.

Various markers of inflammation exist, such as C-reactive protein (CRP), serum amyloid A, fibrinogen, IL-1, IL-6, ICAM-1, selecting, and tumor necrosis factor alpha; at this time, CRP is favored for use in clinical applications (See e.g., Pearson, T. A., et al., Markers of inflammation and cardiovascular disease: application to clinical and public health practice: A statement for healthcare professionals from the Centers for Disease Control and Prevention and the American Heart Association. Circulation, 2003. 107(3): p. 499-511). C-reactive protein (CRP) is an acute-phase reactant that provides a measure of low-grade systemic inflammation. Other markers of inflammation include white blood cell count and VCAM.

Inflammation, as indicated by CRP levels, has been shown to predict future risk of primary and recurrent coronary events independently of other cardiovascular risk factors (Rifai, N., et al., High-sensitivity C-reactive protein: a novel and promising marker of coronary heart disease. Clin Chem, 2001. 47(3): p. 403-11). Inflammation therefore exerts effects that are independent of other risk factors, such as weight. Therefore control of inflammation by calcium may provide health benefits that are independent of the effects of calcium on weight loss. Materials and methods set forth herein for administration of calcium may be used to change a level of inflammation in a subject, and/or to change inflammation marker levels.

Health agencies have begun to recognize a relationship between health and inflammation levels. In recognition of the growing evidence about the health effects of inflammation as evidenced by studies of CRP levels, an expert panel convened by the American Heart Association and the Centers for Disease Control has suggested that high sensitivity(hs)-CRP testing should be considered for patients at intermediate risk of coronary heart disease based on Framingham risk score in order to provide physicians with additional information to guide treatment (Pearson, T. A., et al., p. 499-511). This panel considered hs-CRP levels of <1.0 mg/L low risk, 1.0-3.0 average risk, and >3.0 mg/L high risk. Therefore the use of calcium to control inflammation and inflammation marker levels, e.g., CRP levels, may be beneficial, e.g., a reduction to a level of less than about 1.0 mg/L or less than about 10.0 mg/L, or less than about 3.0 mg/L is useful.

A variety of treatments have been undertaken that suggest that control of inflammation may be an effective treatment for atherosclerosis. Drugs that may reduce inflammation levels as measured by CRP, or attenuate inflammation effects, include insulin sensitizing agents (Chu, N. V., et al., Differential effects of metformin and troglitazone on cardiovascular risk factors in patients with type 2 diabetes. Diabetes Care, 2002.25(3): p. 542-9), aspirin (Ridker, P. M., et al., Inflammation, aspirin, and the risk of cardiovascular disease in apparently healthy men. N Engl J Med, 1997.336(14): p. 973-9), and statins (Strandberg, T. E., et al., Effect of statins on C-reactive protein in patients with coronary artery disease. Lancet, 1999.353(9147): p. 118-9).

Other health studies have implicated certain aspects of nutrition and behavior as affecting inflammation levels. Saturated fat intake was modestly associated with elevated CRP, and dietary fiber was associated with lower CRP in the 1999-2000 National Health And Nutrition Examination Survey (NHANES) from the Center for Disease Control (King, D. E., et al., Relation of dietary fat and fiber to elevation of C-reactive protein. Am J Cardiol, 2003. 92(11): p. 1335-9). Furthermore, a western diet pattern has been correlated with higher CRP levels (Fung, T. T., et al., Association between dietary patterns and plasma biomarkers of obesity and cardiovascular disease risk. Am J Clin Nutr, 2001. 73(1): p. 61-7). Physical activity has also been associated with low CRP levels (Gefflken, D. F., et al., Association between physical activity and markers of inflammation in a healthy elderly population. Am J Epidemiol, 2001. 153(3): p. 242-50) and (Tomaszewski, M., et al., Strikingly low circulating CRP concentrations in ultramarathon runners independent of markers of adiposity: how low can you go? Arterioscler Thromb Vasc Bioi, 2003. 23(9): p. 1640-4). These studies indicate that diet can be effective to change inflammation levels and that administration of a dietary supplement could therefore be a suitable means to adjust inflammation levels.

The administration of calcium to control or reduce inflammation is consistent with other observations regarding physiological effects of calcium. For example, calcium may reduce the amount of trunkal or visceral adipose tissue. Trunkal adipose tissue is adipose tissue generally disposed on the trunk of an animal, and includes adipose tissue disposed in the abdominal area of a human. Visceral adipose tissue is adipose tissue disposed around a body organ. Trunkal and visceral adipose tissue may be significant sources of inflammatory agents, including pro inflammatory cytokines. Adiposity in general may be a source of inflammatory agents, with adiposity tissue correlating to the level of inflammation. Therefore, a treatment that reduces inflammation and also tends to reduce the presence of adipose tissue, including trunkal or visceral adipose tissue, may have a synergistic effect. The administration of calcium may be such a treatment, because administration of effective levels of calcium may not only reduce inflammation levels, but may also tend to reduce a subject's general distribution of adipose tissue, including trunkal adipose tissue.

Also, a diet rich in dairy products may provide calcium that can reduce adiposity without affecting overall body weight (Nocton A. M., et al., Increasing dairy calcium intake reduces adiposity in obese African-American adults. Circulation 2002; 106 (suppl 2) II-610). In one study, 34 otherwise healthy obese African-American adults were maintained on a low calcium (500 mg/day)/low dairy (<1 serving/day) or high dairy (1200 mg Ca/day diet including 3 servings of dairy) with no change in caloric intake or total dietary fat, carbohydrate or protein for 24 weeks (Ibid.). Body weight remained stable for both groups throughout the study, but the amount of body fat was significantly reduced.

Due to a correlation between adiposity and inflammation, the reduction of trunkal and/or visceral adipose tissue may be used as a treatment for reducing inflammation and/or an inflammation marker in a subject. In addition to calcium administration, methods for reducing trunkal and/or visceral adipose tissue include, e.g., a reduced calorie diet, a low-fat diet, a low-carbohydrate diet, high protein diet, high fat diet, low energy density diet, low glycemic index diet, high fiber diet, liquid diet, meal replacement, weight loss pharmaceuticals, an exercise activity, and combinations thereof. These approaches can be combined with the administration of calcium to provide a further reduction in inflammation.

Elevated amounts of adiposity in general, and especially visceral adiposity, can contribute to elevated inflammation levels, as evidenced by measured CRP levels (Tracy, R. P., Is visceral adiposity the “enemy within”? Arterioscler Thromb Vasc Bioi, 2001. 21(6): p. 881-3) and (Lemieux, I., et al., Elevated C-reactive protein: another component of the atherothrombotic profile of abdominal obesity. Arterioscler Thromb Vasc Bioi, 2001. 21(6): p. 961-7). Adipose tissue, and visceral adipose tissue in particular, are regulators of inflammation and serve as a source of proinflammatory cytokines (Tracy, R. P., p. 881-3). These cytokines, e.g., IL-6, induce the synthesis of CRP by the liver (see, e.g., Bataille, R., et al., C-reactive protein levels as a direct indicator of interleukin-6 levels in humans in vivo. Arthritis Rheum, 1992. 35(8): p. 982-4), and visceral fat cells can secrete 2-3 times more IL-6 compared to subcutaneous fat cells (Fried, S. K., et al., Omental and subcutaneous adipose tissues of obese subjects release interleukin-6: depot difference and regulation by glucocorticoid. J Clin Endocrinol Metab, 1998.83(3): p. 847-50). Visceral fat has a strong correlation to CRP in certain subjects (Forouhi, N. G., et al., Relation of C-reactive protein to body fat distribution and features of the metabolic syndrome in Europeans and South Asians. Int J Obes Relat Metab Disord, 2001. 25(9): p. 1327-31). Further, several studies have demonstrated reductions of CRP with weight loss. For example, weight loss of 8 kg over 12 weeks in obese women (BMI 34 kg/m² at baseline) was shown to reduce CRP by 26% (Heilbronn, L. K., et al., Clifton, Energy restriction and weight loss on very-low-fat diets reduce C-reactive protein concentrations in obese, healthy women. Arterioscler Thromb Vasc Bioi, 2001. 21(6): p. 968-70), and a 32% fall in CRP was demonstrated in 25 obese postmenopausal women who lost 15.6% of their body weight (Tchernof, A., et al., Weight loss reduces C-reactive protein levels in obese postmenopausal women. Circulation, 2002. 105(5): p. 564-9).

A reduction in visceral adiposity may be accomplished by reducing trunkal adiposity. Correlations between visceral adipose tissue weight and the weight of subcutaneous adipose tissue of the trunk are significant for both men and women, and total body adipose tissue weight can also correlate with visceral adipose tissue weight (Martin A. D., et al., Relationships between visceral, trunk and whole-body adipose tissue weights by cadaver dissection. Ann Hum Biol. 2003 November-December; 30(6):668-77). CRP concentrations may be elevated in obese individuals who are also insulin resistant and fall in conjunction with weight-loss associated improvements in insulin resistance (see, e.g., McLaughlin, T., et al., Differentiation between obesity and insulin resistance in the association with C-reactive protein. Circulation, 2002.106(23): p. 2908-12). Materials and methods set forth herein for administration of calcium may be used to treat a subject having insulin resistance, e.g., to reduce inflammation and/or to reduce trunkal adipose tissue. Further, such methods may be used in combination with other methods of treating insulin resistance.

The administration of calcium can result in not only a reduction of inflammation, but also a reduction of body fat. An inverse relationship can be observed between calcium intake and adiposity (Parikh, S. J., et al., Calcium intake and adiposity. Am J Clin Nutr, 2003. 77(2): p. 281-7). In a recent human study, an energy-restricted diet containing 1200-1300 mg Ca/day from calcium supplements or dairy sources was compared to the same diet, but with lower calcium intakes (400-500 mg/day). After 24 weeks, both high calcium diets showed significantly greater fat loss overall, and in the trunk area in particular, compared to those on the low calcium diet (Zemel M. B., et al., Dietary calcium and dairy products accelerate weight and fat loss during energy restriction in obese adults. American Journal of Clinical Nutrition, 2002. 75(suppl): p. 342S). An additional 12-week study looked at 2 energy-restricted (−500 cal/day) groups; one group was provided yogurt as the calcium source (1100 mg Ca/day) vs. the control (400-500 mg Ca/day). The yogurt calcium group demonstrated an 81% greater trunk fat loss compared to the lower calcium control (Zemel M. B., et al., Dairy (yogurt) augments fat loss and reduces central adiposity during energy restriction in obese subjects. FASEB Journal, 2003.17: p. Abstract 679.3). Thus, calcium administration generally can be effective to reduce both inflammation as well as body fat.

It may be that weight loss interventions which lead to proportionately greater losses of visceral fat will lead to greater reductions in proinflammatory cytokines and CRP compared to caloric restriction alone. As stated above, visceral adipose tissue is a particularly potent source of inflammatory agents, so that its reduction can be a useful treatment. Further, a reduction of trunkal adiposity typically correlates to a reduction in visceral adiposity. A shift of body composition that causes a reduction in the percentage of body weight is expected to reduce inflammation events, even if total body weight is not decreased, or undergoes a modest increase.

Calcium may be administered to a subject in a therapeutic amount effective to reduce inflammation. Suitable subjects include any animal that can benefit from a reduction of inflammation, and generally includes mammals, such as humans, farm animals and pet animals. The administration may be correlated, in some embodiments, with measurements of inflammation factors. The therapeutic administration may be implemented through food sources and/or dietary supplements. In particular, some food sources may be formulated to provide desired calcium doses for a given quantity of food/caloric intake. Thus, the person may ingest natural calcium sources, food sources enriched with calcium and/or calcium supplements. The efficacy of the therapeutic administration may optionally be evaluated through the monitoring of inflammation markers.

A therapeutic amount will vary between individuals, and may be optimized as needed in the future using techniques known to persons of ordinary skill in these arts. The amount that is effective to reduce inflammation, or an inflammation marker, is considered to be a therapeutic amount. Depending on the current calcium intake of the individual, therefore, additional calcium intake as supplements or dairy foods, or combinations thereof, in the amounts ranging from 100-2000 mg per day are proposed as therapeutic amounts of calcium, with 500-600 mg being a typical dose. Other ranges of intake include, e.g., 10-10,000 mg/day. Other ranges of dosages include 10-10,000 mg. Persons of ordinary skill in these arts will appreciate that all the values and ranges within the ranges explicitly set forth are contemplated. Current governmental recommendations for calcium intake are 1000 mg/day for adults 19-50 years of age, and 1200 mg/day for adults 51 years of age and older. Median calcium intake, however, is lower, and for adult women (ages 31-50) is only 606 mg/day and for adult men (ages 31-50) is only 857 mg/day (Reference: Institute of Medicine. 1997. Dietary Reference Intakes for Calcium, Phosphorous, Magnesium, Vitamin D and Fluoride. Food and Nutrition Board. Washington, D.C.: National Academy Press).

Assuming reductions in visceral fat with calcium would be greater than expected compared to caloric restriction alone, it is expected that CRP reductions would also be greater than weight loss due to caloric restriction alone, especially in conjunction with suboptimal calcium intakes.

Calcium may be administered by providing a food, foods, or food supplement that contain calcium. Examples of such foods include, without limitation, breakfast cereal, snack bars, cereal bars, fruit snacks, bread, dinner rolls, orange juice, meal replacement beverages and bars, salmon, sardines, beans, soymilk, tofu, spinach, turnip greens, kale, broccoli, waffles, pancakes, pizza, milk, yogurt, cheeses, cottage cheese, ice cream, frozen yogurt, nutrient supplements, calcium fortified vitamin supplements, and liquids supplemented with calcium.

Techniques that are known to help to enhance calcium absorption may be used in combination with administration of calcium and/or reduction of inflammation. Materials and methods to accomplish such techniques may, for example, be provided with a calcium source, provided as part of an instruction in a kit, or incorporated into other techniques described herein. The absorption of calcium is known to be increased, for example, through the action of 1,25-dihydroxyvitamin D, also known as the active form of vitamin D (Reference: Institute of Medicine. 1997. Dietary Reference Intakes for Calcium, Phosphorous, Magnesium, Vitamin D and Fluoride. Food and Nutrition Board. Washington, D.C.: National Academy Press). Also, inulin and other fermentable soluble fibers increase calcium absorption (Zafar T. A., et al. Nondigestible oligosaccharides increase calcium absorption and suppress bone resorption in ovariectomized rats. J Nutr. 2004 February; 134(2):399-402). In addition, any supplemental form of calcium may be provided in a form that will disintegrate for improved absorption. Doses of 500 mg or less tend to be more efficiently utilized (Reference: Institute of Medicine. 1997. Dietary Reference Intakes for Calcium, Phosphorous, Magnesium, Vitamin D and Fluoride. Food and Nutrition Board. Washington, D.C.: National Academy Press).

At least one dairy product may be administered as a source of calcium. A dairy product is a product having milk, a milk derivate, or a material that is derivable from milk. Examples of dairy products are milk, cheese, cottage cheese, yogurt, butter, and cream. Another example of a dairy product is a protein or factor concentrated and/or isolated from milk, e.g., a dairy protein isolate.

A calcium supplement may be administered as a source of calcium. Examples of calcium supplements include calcium salts, e.g., CaCO₃, CaSO₄, and CaCl₂. Supplements may be in, e.g., in solid, liquid, or mixed solid-liquid forms. Examples include pills, powders, and dehydrated mixtures.

The administration of calcium can lead to a change in inflammation in a mammal, including a change in the level of an inflammation marker in a mammal, e.g., in its tissue or bodily fluid. Tissue refers to a part of an organism consisting of an aggregate of cells having a similar structure and function, and includes, e.g., an organ, liver, artery, a portion of extracellular matrix, adipose tissue, connective tissue, muscle, nerve, blood, and bone. Bodily fluid refers to any fluid or gas derived from a body, including saliva, urine, blood, serum, tears, lymph, mucus, plasma, and exhalates.

In some embodiments, the selection of a therapeutic amount of calcium can be based on achieving a selected change in an inflammation level or marker. For example, one embodiment is a method for modifying a biological condition of a mammal that involves administering a therapeutic amount of calcium to the mammal, with the therapeutic amount of calcium being selected to change a level of inflammation in the mammal. The selection of the calcium, and/or the selection of the amount of calcium, is caused by the discovery that calcium affects levels of inflammation and/or inflammation markers.

An aspect of certain embodiments involves measuring the level of an inflammation marker for a mammal, for example, in association with administering calcium. Assessments may be performed using a sample taken from the mammal, for example, a sample of a tissue or a bodily fluid, e.g., a sample of blood, urine, saliva, or adipose tissue. Examples of calcium administration are set forth herein. An inflammation level may be measured, before, during, and/or after administration of calcium. The calcium level may be adjusted upwards or downwards, or not changed, in response to measured inflammation levels. For example, a baseline CRP level may be measured in a subject and then remeasured during the course of a treatment of calcium, and the calcium level adjusted upwards if a desired CRP level is not obtained.

Selection of a subject for treatment in accordance with methods of the invention may, in some embodiments, relate to a condition of the subject. The selection criterion, or criteria, may be based on a general recommendation for that species, e.g., for all humans, or may further be based on particular characteristics, e.g., gender, weight, age, health condition, genetic heritage, or body fat. Alternatively, the selection may be based on a health history of the subject, e.g., suffering from a particular condition, or being the subject of particular risk factors. In some embodiments, a subject is selected because it has a condition of obesity, excess body fat, or an inflammatory condition.

One condition that may be suitable for selecting a subject for treatment is obesity. Obesity, for a human, is a condition of having a body mass index (BMI) of at least about 30. Being overweight refers to a human having a body mass index of at least about 25. BMI is a tool for indicating weight status, and is a measure of weight for height expressed as kg/m². BMI correlates with body fat, and this relationship may differ with age and gender. Women are more likely to have a higher percent body fat than men for the same BMI, and older individuals tend to have more body fat than younger individuals with the same BMI (per the guidelines described in “Clinical Guidelines on the Identification, Evaluation, and Treatment of Overweight and Obesity in Adults;” Bethesda, Md.: NHLBI, 1998).

Another condition that may be suitable for selecting a subject for treatment is the existence of an inflammatory condition in the subject. Examples of inflammatory conditions are atherosclerosis and other cardiovascular diseases for which inflammation contributes. There are a variety of other inflammatory conditions, including atherosclerosis, arteriosclerosis, diabetes, arthritis, gum disease, infection, rheumatoid arthritis, psoriasis, asthma, systemic lupus erythematosus, and osteoarthritis.

Calcium treatments as described herein are also expected to benefit subjects having metabolic syndrome. Metabolic syndrome is characterized by a group of risk factors: trunkal adiposity, atherogenic dyslipidemia (blood fat disorders), raised blood pressure (about 130/85 mmHg or higher), insulin resistance or glucose intolerance, prothrombotic state (e.g., high fibrinogen or plasminogen activator inhibitor 1), and proinflammatory state (e.g., elevated CRP in the blood). The presence of at least one, two, or three of these factors may be a basis for selecting a subject for treatment, e.g., insulin resistance and/or elevated CRP. The underlying causes of this syndrome are believed to be related to trunkal adiposity and overall excess weight, physical inactivity, and some genetic factors. Metabolic syndrome has been found to be associated with an increased risk of coronary heart disease, diseases related to plaque buildups in artery walls, and type 2 diabetes. Waist circumference measurements are considered a risk factor for metabolic syndrome. The risk can be higher for men having a waistline of more than about 102 cm (40 in), and for women having a waistline of more than about 88 cm (35 in). Since calcium administration can affect both inflammation and trunkal adiposity, it is expected to be useful for treating metabolic syndrome. Metabolic syndrome is further discussed in the Third Report of the National Cholesterol Education Program (NCEP) Expert Panel on Detection, Evaluation, and Treatment of High Blood Cholesterol in Adults (Adult Treatment Panel III) Final Report, NIH Publication No. 02-5215 September 2002, Circulation. 2002; 106:3143-3421.

As described herein, clinical studies have been designed to demonstrate the control of inflammation in a subject using calcium and/or various calcium sources. Some studies could be performed using adults that have a particular condition, e.g., obesity, metabolic syndrome, or being overweight. For example, about eighty (80) obese, but otherwise healthy, men and women (average baseline BMI about 34 kg/m2) with an average CRP of about 6.0 mg/L at baseline could be randomized to about 2 groups for about a 12 week intervention. Both groups would consume reduced calorie diets (about −500 kcal) to promote weight loss, with the calcium intake in “the control group” adjusted to about 500 mg/day and in “the intervention group” adjusted to about 1200 mg/day using a calcium source. Examples of calcium sources are calcium carbonate, calcium citrate, calcium chloride, tricalcium phosphate, calcium carbonate, and/or dairy sources including milk, yogurt, and/or cheese. DEXA and hs-CRP measurement can be performed at baseline and about 12 weeks. It is expected that significantly greater loss of visceral fat (on the order of about 50% of fat loss from trunkal region in calcium carbonate group vs. about 20% of fat loss from trunkal region in control group) could be demonstrated in the “intervention group” with a corresponding reduction in CRP greater than the control group.

Other clinical studies could be performed over a longer period of time and/or with relatively larger cohorts of subjects. For example, about 1000 obese, insulin resistant men and women (average BMI about 35 kg/m²) with elevated CRP (about more than 3.0 mg/L at baseline) could be randomized to two groups, one with no calcium addition to the typical diet, and the other with about 1000 mg/day additional calcium as calcium carbonate and mixed dairy sources. Time measurements could include, e.g., CRP, atherosclerosis progression, fasting glucose, and cardiovascular events. It is expected that the higher calcium group would have lower CRP, reduced atherosclerosis, fewer cardiovascular events, and fewer cases of type 2 diabetes.

Some embodiments include a material and/or method of using calcium as set forth herein in combination with another treatment such as exercise or diet. An example of a diet treatment is a reduced calorie diet, a low calorie diet, a low carbohydrate diet, a low fat diet, and a diet for a person having diabetes or insulin resistance. An example of an exercise treatment is a program of physical exercise involving a heightened time of cardiovascular or muscular activity.

Kits may be prepared to provide for administration of calcium to effectuate a treatment as described herein, e.g., control of inflammation. A kit may include a calcium source and an instruction for its administration. Exemplary instructions include methods of administration, e.g., with respect to quantity, sources, or timing of administration of a calcium source. Instructions may also, or alternatively, include other aspects of treating a subject in conjunction with calcium administration, e.g, as related to diet or exercise. Kits are useful for providing a calcium source and an instruction together so as to help a user practice a process of calcium administration that is geared for their use, and/or to practice related regimens to derive additional benefits from the calcium source.

Some embodiments of the invention include providing an instruction for administration of calcium, in combination with another treatment such as exercise or diet. As used herein, instruction includes any teaching, and may be in written, electronic, or other form of communication. Examples of an instruction suitable for kits include, but are not limited to, a book, an audiovisual presentation, a slide, a brochure, a web site, an audio presentation, a label, and an advertisement. When used in the form of a book, the instruction can detail a particular diet treatment and thereby provide an instruction related thereto. Another example is a food container having a writing on or in the container that describes selecting the food product for calcium intake to treat inflammation and/or visceral adiposity. A further example is a food container indicating a further source of communication, such as a website, which can contain instructions for administration of calcium and/or reduction of adiposity and/or visceral adiposity.

Specific embodiments and examples have been used herein; however, these embodiments are not intended to limit the scope or spirit of the invention. All patents, patent applications, and publications referenced herein are hereby incorporated herein by reference.

1. A method of modifying a biological condition of a subject, comprising:
administering a therapeutic amount of calcium to the subject, wherein the therapeutic amount of calcium is selected to change a level of inflammation in the subject.

2. The method of claim 1, wherein the level of inflammation in the subject is changed in a tissue or bodily fluid of the subject chosen from the group consisting of blood, plasma, serum, lymph, saliva, urine, liver, artery, adipose tissue and mixtures thereof.

3. The method of claim 1, further comprising: selecting the amount of calcium to reduce a level of an inflammation marker for the subject.

4. The method of claim 3, wherein the inflammation marker is C-reactive protein, and the C-reactive protein level is reduced to less than about 10 mg/L of C-reactive protein in the subject.

5. The method of claim 3, wherein the inflammation marker is at least one member of the group consisting of C-reactive protein, serum amyloid A, IL-1, IL-6, tumor necrosis factor alpha, fibrinogen, white blood cell count, vascular cell adhesion molecule (“VCAM”), intercellular adhesion molecule (“ICAM”), and selecting.

6. The method of claim 1, wherein the administering of calcium comprises ingesting at least one calcium containing product chosen from the group consisting of dairy products, dairy protein isolates, dietary supplements, foodstuffs supplemented with calcium, foods high in calcium, and calcium fortified cereal.

7. The method of claim 1, wherein administering the calcium to the subject is effective to produce a decrease in adiposity of the subject.

8. The method of claim 1, wherein administering the calcium to the subject is effective to produce a decrease in trunkal adiposity of the subject.

9. The method of claim 1 wherein the subject has a condition of insulin resistance.

10. The method of claim 1, wherein the subject has a condition of being overweight, as indicated by a Body Mass Index of greater than 30.

11. The method of claim 1, wherein the subject has an inflammatory condition and wherein the inflammatory condition is chosen from the group consisting of atherosclerosis, arteriosclerosis, diabetes, arthritis, gum disease, infection, psoriasis, asthma, systemic lupus erythematosus, and combinations thereof.

12. The method of claim 1, further comprising providing the subject with an instruction for a weight loss protocol and wherein the instruction comprises a member of the group consisting of a writing, a book, an audiovisual presentation, a slide, a brochure, a web site, an audio presentation, a label, and an advertisement.

13. A method of reducing inflammation in a subject comprising:
measuring a C-reactive protein level in the subject; and
administering a calcium source to the subject to induce a change in the C-reactive protein level.

14. The method of claim 13, wherein the subject has a condition of insulin resistance, obesity, or being overweight.

15. A kit for modifying a biological condition of a subject, comprising:
a source of calcium; and
an instruction for administering the source of calcium to the subject to change a level of inflammation in the subject.

16. The kit of claim 15, wherein the instruction further comprises a description of administering the source of calcium to the subject to reduce a level of the inflammation marker in the subject.

17. The kit of claim 15, wherein the inflammation marker is at least one member of the group consisting of C-reactive protein, serum amyloid A, IL-1, IL-6, tumor necrosis factor alpha, fibrinogen, white blood cell count, VCAM, ICAM, and selecting.

18. The kit of claim 15, wherein the source of calcium comprises at least one member of the group consisting of dairy products, dairy protein isolates, dietary supplements, foodstuffs supplemented with calcium, foods high in calcium, calcium fortified cereal, breakfast cereal, snack bars, cereal bars, fruit snacks, bread, dinner rolls, orange juice, meal replacement beverages and bars, salmon, sardines, beans, soymilk, tofu, spinach, turnip greens, kale, broccoli, waffles, pancakes, pizza, milk, yogurt, cheeses, cottage cheese, ice cream, frozen yogurt, nutrient supplements, calcium fortified vitamin supplements, and liquids supplemented with calcium.

19. The kit of claim 15, wherein the instruction further comprises a description of administration of the source of calcium to the subject to produce a decrease in adiposity of the subject.

20. The kit of claim 15, wherein the instruction further comprises a weight loss protocol.
